(19) United States
(12) Patent Application Publication
(10) Pub. No.: US 2011/0160272 A1
(43) Pub. Date: Jun. 30, 2011

(54) METHODS AND COMPOSITIONS FOR USE OF ANGIOGENESIS INHIBITORS IN THE PREVENTION AND/OR CONTROL OF EPILEPSY

(76) Inventor: KENNETH A. JENROW, Ferndale, MI (US)

(21) Appl. No.: 12/981,257

(22) Filed: Dec. 29, 2010

Related U.S. Application Data
(63) Continuation of application No. 11/575,458, filed on Mar. 16, 2007, now abandoned.
(60) Provisional application No. 60/610,936, filed on Sep. 17, 2004.

(51) Int. Cl. A61K 31/404; A61K 31/40; A61K 31/395; A61P 9/00; A61P 25/00

(52) U.S. Cl. 514/414; 514/423; 514/183

(57) ABSTRACT

In accordance with some preferred embodiments, without limitation, the invention comprises methods and compositions for the prophylactic and/or antiepileptic administration of angiogenesis inhibitors in conjunction with seizures or traumatic insults to the brain, which administration may limit the extent of trauma-associated angiogenesis and/or decrease the likelihood that angiogenesis will give rise to regional epileptogenecity by preventing/reducing the associated increases in blood-brain barrier permeability among new or existing blood vessels along with their epileptogenic effects.

### PRIORITY CLAIM

This application is a continuation application claiming priority to U.S. National Phase application Ser. No. 11/575,458 filed Mar. 16, 2007, which claims priority to International Application No. PCT/US05/33340 filed Sep. 19, 2005, which claims priority to U.S. Provisional Patent Application No. 60/610,936 filed Sep. 17, 2004, the complete disclosures of which are each hereby incorporated in full.

### FIELD OF THE INVENTION

The present invention relates to the field of methods and compositions for treatment of neurological conditions.

### BACKGROUND

Epilepsy is the most prevalent major neurological disorder, affecting approximately 1% of the U.S. population (1). Prolonged bouts of limbic status epilepticus (“SE”) are commonly associated with the development of temporal lobe epilepsy (“TLE”) in humans, and are widely used as a means of creating epilepsy in animal models. Although relatively few individuals who experience SE go on to develop TLE (2, 3, 4), retrospective studies of adults with TLE demonstrate a high prevalence (>75%) of a prolonged bout of SE earlier in life (5, 6). Epilepsy is also associated with other forms of neurotrauma such as neoplasms (tumors), head injury, hypoxia-ischemia (stroke), and encephalitis, all of which increase the risk ratio of developing epilepsy by more than an order of magnitude (1).

While the precise mechanisms by which these pathologies promote epileptogenesis remain obscure, they have all been associated with the selective depletion of neurons, gliosis, and the development of angiogenesis and/or chronic changes in the permeability of the blood brain barrier (“BBB”).

Many of the blood vessels formed during trauma-associated angiogenesis are known to have abnormal BBB function, which promotes the aberrant leakage of materials, normally sequestered in the plasma, into the interstitial space. These include small plasma-borne proteins, peptides, amino acids, and assorted ion species (7). Some of these molecules and ions may adversely affect brain cells and promote epileptogenesis. This could involve direct effects on neuronal excitability or indirect effects resulting from changes in tissue osmolarity. For example, glutamate release associated with stroke has recently been shown to produce chronic alterations in intracellular calcium that were epileptogenic (8), whereas osmotically-induced reductions in extracellular space have been shown to enhance both excitatory synaptic transmission and field (ephaptic) effects in the neocortex, and could therefore promote the recruitment and neuronal synchrony characteristic of epileptiform activity (9).

Although scientific literature exists in which angiogenesis inhibitors have been applied to control tumor growth and to limit damage to the brain resulting from stroke and impact trauma (10, 11, 12), none of these papers have addressed the effects of such treatment on seizure expression associated with these insults. Similarly, these compounds have never been applied as means of treating trauma associated with prolonged SE, or spontaneous seizures once they are already established. Thus, an unmet need exists for therapeutic treatments to prevent, control, or alleviate epileptic symptoms by affecting mechanisms of angiogenesis and related indicia, including without limitation, blood vessel formation and/or leakage.

### SUMMARY

The present invention meets this unmet need by comprising novel methods and compositions to prevent, control, or alleviate epilepsy through the selective application of angiogenesis inhibitors. In accordance with some preferred embodiments, without limitation, the invention comprises methods and compositions for the prophylactic and/or antiepileptic administration of angiogenesis inhibitors in the wake of a traumatic insult to the brain which may limit the extent of trauma-associated angiogenesis and/or decrease the likelihood that angiogenesis will give rise to regional epileptogenecity by preventing/reducing the associated increases in BBB permeability among new or existing blood vessels along with their epileptogenic effects.

Growth factors are known to participate in trauma-associated angiogenesis. One such growth factor is vascular endothelial growth factor (“VEGF”), which is also known to promote increased microvascular permeability even in the absence of angiogenesis. Angiogenesis inhibitors that antagonize VEGF signaling, or otherwise block or ameliorate the effectuation of VEGF, are therefore of particular interest, because chronic VEGF production among reactive astrocytes could perpetuate (and be perpetuated by) continual insults arising from spontaneously recurring seizures, comprising an epileptogenic cycle. This cycle may be broken, even in established epilepsy, by administering VEGF inhibitors for an interval sufficient to permit the resolution of reactive astrocytosis and its associated VEGF production and/or the selective regression of abnormal and leaky neovessels.

In contrast to other forms of therapy, in accordance with the invention, there is a high likelihood that the duration of drug therapy would be relatively brief and with a high probability of success. Prophylactic administration of angiogenesis inhibitors may greatly reduce the incidence of epilepsy associated with many forms of neural trauma, including SE. Administration of the appropriate angiogenesis/growth factor inhibitors where epilepsy is already established may be similarly effective, and may offer a new line of attack for treating epilepsy without the deleterious side effects associated with current drug therapies, or where current drug therapies have failed.

Other aspects of the invention will be apparent to those skilled in the art after reviewing the detailed description below.

### BRIEF DESCRIPTION OF THE DRAWINGS

The present invention will now be described, by way of example only and without limitation, with reference to the accompanying drawings, in which:

FIG. 1 shows composite EB-Alb and FITC-dextran images acquired using a fluorescent microscope from 100 μm coronal rat brain sections obtained from A) spontaneously epileptic animals 12 weeks after KA-induced SE, or B) from nonepileptic age matched control animals.

FIG. 2 shows confocal EB-Alb and FITC-dextran images acquired from coronal sections at 10X (top row) and 40X (bottom row), from the ventral hippocampus of the rat brain 8 weeks after KA-induced SE (right column), together with corresponding images from an age matched control (left column).

FIG. 3 shows confocal EB-Alb and FITC-dextran images acquired from horizontal sections at 10X (top row) and 40X (bottom row), from the ventral hippocampus of the rat brain 16 weeks after KA-induced SE (right column), together with corresponding images from an age matched control (left column).

FIG. 4 shows autoradiographs obtained from coronal rat brain sections harvested twelve weeks after A) KA-induced SE, or B) saline injection in an age matched control.

FIG. 5 shows fluorescent and light microscopic images of VEGF staining acquired at 40X in the ventral hippocampus from coronal rat brain sections.

FIG. 6 shows light microscopic images of BrdU staining acquired at 20X in the amygdala from coronal rat brain sections.

FIG. 7 is a graph showing observed cumulative spontaneous seizures among KA-induced SE and control rats.

### DETAILED DESCRIPTION

In some preferred embodiments, without limitation, the present invention comprises novel methods and compositions for the selective administration of angiogenesis inhibitors in conjunction with the treatment of traumatic insults to the mammalian brain, for purposes of limiting the extent of trauma-associated angiogenesis and decreasing the probability that angiogenesis and/or changes in vascular permeability associated with the insult will be epileptogenic. In general, angiogenesis inhibitors exert their effects by blocking a set of biochemical and cellular responses/processes common to a variety of growth factors known to promote angiogenesis. More specific angiogenesis inhibitors target particular proangiogenic growth factors by interfering with the synthesis of the growth factor or its receptor(s), by selectively removing (scavenging) the growth factor after its release, or by selectively blocking growth factor-specific receptors or their associated biochemical/enzymatic pathways. Blocking these responses/processes at any point limits the extent of post-traumatic angiogenesis. Thus, for purposes of the invention, angiogenesis inhibitors may comprise any chemical or biological molecule which blocks or limits the induction or effectuation of intra- or inter-cellular proangiogenic growth factor influence. These include, without limitation, the synthesis of a proangiogenic growth factor or its receptors, the bioavailability of a proangiogenic growth factor, the binding of a proangiogenic growth factor to its receptor, biochemical/enzymatic pathways initiated by such growth factors, or the expression or action of proteins involved in angiogenesis including cell proliferation, cell motility, and interactions involving the extracellular matrix.

Vascular endothelial growth factor (“VEGF”) is known to be a major growth factor that participates in post-traumatic angiogenesis; however, it is also unique among such growth factors in that it can additionally promote increased microvascular permeability in the absence of angiogenesis i.e., via its effects on preexisting microvessels. Moreover, microenvironmental elevation of VEGF concentration above a particular threshold in otherwise normal tissue is sufficient to induce the formation of abnormal and leaky microvessels, characteristic of post-traumatic angiogenesis, which selectively regress if VEGF concentrations subsequently fall below this threshold (13). In some embodiments, without limitation, the invention comprises the selective administration of angiogenesis inhibitors that specifically antagonize VEGF signaling to produce efficacious results, in part because chronic VEGF production by reactive astrocytes may perpetuate (and be perpetuated by) continual insults arising from spontaneously recurring seizures associated with established epilepsy, comprising an epileptogenic cycle. In accordance with the invention, this cycle may be broken by administering angiogenesis inhibitors for an interval sufficient to permit the selective regression of abnormal and leaky neovessels and/or the resolution of reactive astrocytosis and its associated VEGF production.

Thus, prophylactic administration of angiogenesis inhibitors comprising some embodiments may greatly reduce the incidence of epilepsy associated with many forms of neural trauma by limiting the associated angiogenesis and increases in BBB permeability, along with their effects on epileptogenesis. Similarly, in some embodiments, antiepileptic administration of angiogenesis inhibitors to break a growth factor-mediated epileptogenic cycle, where epilepsy is already established, may also be an effective treatment for epilepsy without the deleterious side effects associated with current drug therapies, or where current drug therapies have failed.

SE and other forms of neural trauma cause injuries to structures in the brain initiated by a cascade of events that are the subject of continuing investigation and debate. It is known, however, that every form of neurotrauma has hallmark manifestations, which include neuronal depletion, gliosis, and the development of angiogenesis and/or increased microvascular permeability.

Epilepsy is often associated with prolonged bouts of SE which occur commonly during childhood in response to high fever, otherwise known as “febrile seizures”. Epilepsy is also commonly associated with other forms of acute neural trauma such as head impact, encephalitis, and hypoxia-ischemia (stroke), or as a secondary phenomenon accompanying chronic neurological conditions such as neoplasms (tumors) or arteriovenous malformations.

Gliosis, characterized by the presence of reactive astrocytes, is a prominent feature in virtually every seizure foci, and reactive astrocytes are a primary source of VEGF in regions of neural trauma. Along with other growth factors, VEGF promotes angiogenesis in the wake of a traumatic insult to the brain. The resultant neovessels typically lack normal BBB function, such that they generally exhibit increased microvascular permeability. VEGF is unique among growth factors in that it also promotes increased microvascular permeability among capillaries not associated with post-traumatic angiogenesis. Such compromises in BBB permeability may play a role in epileptogenicity. As one example, without limitation, there may be an excitatory neurotransmitter in plasma, such as the excitatory neurotransmitter glutamate, which leaks across the BBB into the interstitium and biases the traumatized tissue toward excitability. Alternatively, plasma proteins may escape into the interstitium, where they are taken up by neurons and glia, rendering them hyperosmotic. The resultant swelling of these cells can promote excitatory neurotransmission and the development of neuronal synchrony and recruitment characteristic of seizure onset.

In our model of epileptogenesis, neural trauma is followed by a period of angiogenesis that leads to the production of neovessels lacking normal blood brain barrier function, and exhibiting increased microvascular permeability. Leakage of materials across the BBB in these regions promotes focal hyperexcitability and the development of epileptogenesis. Once spontaneous seizures are initiated, they may perpetuate the accompanying gliosis and VEGF production. This VEGF production may then promote or sustain leaking in neovessels and/or other capillaries (i.e. preexisting capillary beds), along with the occurrence of spontaneous seizures.

BBB leakage associated with neural trauma is often found in areas of the brain that are highly integrated, such as the limbic system and other areas involved in propagating and initiating seizures. Once a region of focal epileptogenicity is established, seizures can propagate outward within these networks using preexisting neuronal pathways to involve otherwise normal brain structures. This tendency to propagate often increases with repeated seizure activity. This phenomenon is illustrated in animal models by a process known as kindling, in which the experimental delivery of brief electrical stimulation to the brain, gives rise to seizures of increasing severity over time. There is good evidence from the kindling model that these brief repetitive seizures represent a form of repetitive neural trauma, capable of sustaining gliosis and VEGF production; however, if kindling is discontinued for a period of approximately two months, the accompanying gliosis and VEGF production generally subside back to baseline levels.

Thus, in some preferred embodiments, the invention comprises novel methods to prevent, control, or alleviate epilepsy through the selective application of appropriate angiogenesis inhibitors. In accordance with some embodiments, without limitation, one may inhibit angiogenesis following trauma to the brain, such as in head impact, hypoxia-ischemia (stroke), neoplasms, infections, or febrile seizures, through the prophylactic administration of one or more angiogenesis inhibitors for a finite interval of time, thereby limiting the development of angiogenesis and the associated increases in BBB permeability, and reducing likelihood of developing epilepsy later on.

In accordance with some embodiments, without limitation, the invention comprises methods for selective application of one or more angiogenesis inhibitors that act as blockers of VEGF signaling. Thus, in established epilepsy, with angiogenesis, leakage, gliosis, VEGF production, and spontaneous seizures, application of an angiogenesis inhibitor targeting VEGF would result in the selective recession of abnormal and leaky microvessels and/or reduce or eliminate the leakage of capillary beds produced by the continuing VEGF production. The cessation of seizures resulting from this intervention, perhaps in combination with other established antiepileptic drugs, would permit the gliosis and VEGF production to subside, bringing seizures to a permanent halt. Thus, in established epilepsy, one would use this form of antiepileptic therapy to initially neutralize the sustaining influence of VEGF on pathological neovessels and BBB permeability, halt the occurrence of spontaneous seizures, and ultimately permit VEGF production among reactive astrocytes to resolve, thereby breaking the epileptogenic cycle.

In some embodiments, without limitation, the invention comprises the selective administration of one or more angiogenesis inhibitors that impede the synthesis, bioavailability, or effects of proangiogenic growth factors or their receptors, including without limitation, VEGF. As one example only, application of an angiogenesis inhibitor which blocks growth factor mediated activation of tyrosine kinase would block all subsequent steps in the enzymatic cascade and prevent the angiogenic influence of VEGF. Thus, in accordance with the invention, angiogenesis inhibitors that disrupt tyrosine kinase-mediated signaling, as a class, would prevent angiogenesis and the formation of neovessels, which are preferentially leaky, and/or prevent or mitigate the effects of growth factors in producing increased permeability of existing blood vessels.

Similarly, in some embodiments, the invention comprises the selective administration of angiogenesis inhibitors that act by interacting with or blocking other steps in the biochemical effectuation of intra- or inter-cellular growth factor influence, including without limitation, VEGF influence. As two examples only, without limitation, such activity may include interacting with or disturbing the activity of integrins and/or other extracellular matrix proteins.

For purposes of the invention, angiogenesis inhibitors may comprise, without limitation, any chemical or biological molecule which blocks or limits the synthesis, bioavailability, or effectuation of intra- or inter-cellular proangiogenic growth factors, including the binding of such growth factors to receptors and any resultant biochemical, enzymatic, or cell mediated responses pertinent to angiogenesis and/or vascular permeability. There are numerous established and developing approaches for inhibiting angiogenesis associated with tumor growth and all of these are considered candidates for use, either alone or in combination, in antiepileptogenic or antiepileptic therapy in accordance with the invention. These include, without limitation, naturally occurring angiogenesis inhibitors (e.g., angiostatin, endostatin, thrombospondins, platelet factor-4, etc.) delivered either systemically or in a targeted fashion (e.g. stem cells), inhibitors of endothelial cell growth or proliferation (e.g., TNP-470, thalidomide, interleukin-12, combretastin A4, etc.), inhibitors of proangiogenic molecules including antibodies, antisense and soluble receptors for VEGF and FGF (e.g., Avastin, VEGF-trap, NM-3, etc.), agents that interfere with basement membranes and extracellular matrix (BMS-275291, tissue inhibitors of matrix metalloproteases [TIMPs], etc.), antibodies to or inhibitors of adhesion molecules (e.g., Vitaxin, Cilengitide, etc.), small molecule inhibitors of receptor tyrosine kinases (SU5416, SU6668, SU11248, etc.), COX-2 inhibitors (e.g., Celecoxib), RNA interference for post-transcriptional gene silencing, antiangiogenic gene therapy (e.g., Thrombospondin-1, Endostatin, Angiostatin, Vastatin, etc.) delivered by nonviral or viral vectors (e.g., plasmid DNA, cationic liposomes, antisense RNA, small interfering RNA, adenoviruses, retroviruses, lentiviruses, herpies simplex, etc.), targeted antiangiogenic gene therapy (using vascular targeting agents, phage vectors, nanoparticles, etc.) (14).

Increases in microvascular permeability may be specific to neovessels formed during angiogenesis, which typically occurs acutely following an insult to the brain. In this instance, prophylactic treatment with angiogenesis inhibitors during this acute post-traumatic interval may impede angiogenesis and the associated increase in microvascular permeability, and prevent the subsequent development of epilepsy. Alternatively, the growth factor milieu in established regions of epileptogenesis, perhaps perpetuated by the repetitive insults associated with spontaneously recurring seizures, may be required to sustain the abnormal and leaky neovessels and/or promote increases in microvascular permeability among otherwise normal preexisting capillaries. In this instance, antiepileptic treatment with the appropriate angiogenesis/growth factor inhibitors may be sufficient to break this cycle, and reduce the occurrence of spontaneously recurring seizures where they are already well established.

Thus, in accordance with the invention, there is a high likelihood that the duration of drug therapy would be relatively brief and with a high probability of success. Prophylactic administration of efficacious amounts of angiogenesis inhibitors may greatly reduce the incidence of epilepsy associated with many forms of neural trauma. Antiepileptic administration of the appropriate angiogenesis inhibitors in efficacious amounts, where epilepsy is already established, may be similarly effective and may offer a new line of attack for treating epilepsy without the deleterious side effects associated with current drug therapies, or where current drug therapies have failed.

In accordance with the invention, the preferred route of administration of angiogenesis inhibitors in humans is by oral administration. However, any appropriate routes of administering such inhibitors known to those of ordinary skill in the art also comprise embodiments of the invention.

Some disparity exists between the latent period observed in animal models of epilepsy, where the latent period following chemically induced status epilepticus is approximately two weeks on average, and that observed clinically following a specific insult to the human brain, where the average latent period is approximately 7.5 years. This suggests that in humans, there is either a relatively prolonged period of epileptogenesis or that a “second hit”, in the form of some genetic and/or environmental factor, is additionally required for the development of epilepsy. The data so far favors the second hit hypothesis. According to this hypothesis, an ‘initial precipitating insult’ results in pathological changes that lower seizure threshold, after which a second hit results in the expression of epilepsy. This hypothesis is supported by observations suggesting that the rates for the development of neuronal, glial, and vascular pathologies do not differ appreciably from those observed in animal models following similar insults. Moreover, unlike animal models of epilepsy, relatively few patients who experience such insults proceed to develop epilepsy.

Since the use of angiogenesis inhibitors in accordance with the invention specifically targets the evolution and expression of vascular pathologies, it is expected that the timing and duration of treatment in humans will approximate those established for animal models following status epilepticus or other forms brain insult. Similarly, the doses established for achieving antiangiogenesis using such compounds in animal epilepsy models, or for other clinical applications in humans (as one example only, for cancerous tumors), would be expected to be applicable in this context as well. (15)

The angiogenesis inhibitor(s) of the present invention is administered and dosed in accordance with good medical practice, taking into account the clinical condition of the individual patient, the site and method of administration, scheduling of administration, patient age, sex, body weight and other factors known to medical practitioners. The “pharmaceutically effective amount” for purposes herein is thus determined by such considerations as are known in the art. The amount must be effective to achieve improvement, including but not limited to, decreased indicators of angiogenesis and vascular permeability, decreased seizure frequency or severity, or improvement or elimination of symptoms and other indicators as are selected as appropriate measures by those skilled in the art.

In accordance with the present invention, the angiogenesis inhibitor(s) can be administered in various ways. It can be administered alone or as an active ingredient in combination with pharmaceutically acceptable carriers, diluents, adjuvants and vehicles. The angiogenesis inhibitor(s) can be administered orally, subcutaneously or parenterally including intravenous, intraarterial, intramuscular, intraperitoneal, and intranasal administration as well as intrathecal and infusion techniques, or by local administration or direct inoculation to the site of disease or pathological condition. Implants of the compounds are also useful. The patient being treated is a warm-blooded animal and, in particular, mammals including humans. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the experimental animals exemplified herein which treatment has a length proportional to the length of the disease process and drug effectiveness. The doses may be single doses or multiple doses over periods of time. The treatment generally has a length proportional to the length of the disease process and drug effectiveness and the patient species being treated.

When administering the angiogenesis inhibitor(s) of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

When necessary, proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for angiogenesis inhibitor(s) compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the angiogenesis inhibitor(s).

Sterile injectable solutions can be prepared by incorporating the angiogenesis inhibitor(s) utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the angiogenesis inhibitor(s) utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Many other such implants, delivery systems, and modules are well known to those skilled in the art.

In some embodiments, without limitation, the angiogenesis inhibitor(s) of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered and timing of administration may vary for the patient being treated.

Use of prophylactic and antiepileptic approaches comprising the invention may greatly reduce the incidence of epilepsy and the costs associated with the chronic management of this condition, and with minimal associated side effects. Where epilepsy is already established, the invention may provide a means of controlling epilepsy with minimal side effects, reducing the need for the costly management of patients currently medicated with drug “cocktails” consisting of multiple anticonvulsants. Moreover, embodiments of the invention may offer a means of treating forms of epilepsy that are refractory to existing drug therapies, thereby reducing the need for costly surgical interventions and, for some, offering a means of treatment where none presently exists.

### EXAMPLES

The following examples of embodiments of the invention are provided without limiting the scope of the invention to only those described below.

Human temporal lobe epilepsy (TLE) is typically associated with the occurrence of a prolonged episode of limbic SE that results in a characteristic pattern of damage in the hippocampus and other medial temporal structures. This manifests as a pattern of neuronal depletion, synaptic reorganization, and gliosis (reactive astrocytosis), referred to as mesial sclerosis, that evolves during a seizure-free latent period separating SE from the emergence of spontaneous recurring seizures (SRS), which in humans can be months to years in duration. A similar progression occurs in animal models of TLE, where a prolonged episode of limbic SE is induced experimentally, resulting days to weeks later in the development of SRS of limbic origin, along with mesial sclerosis that is virtually identical to that associated with human TLE. Angiogenesis/VEGF inhibitors administered during the latent period may prevent the associated increase in BBB permeability, possibly averting the emergence of SRS as a result (prophylactic therapy). Angiogenesis/VEGF inhibitors administered once spontaneously recurring seizures are already established may reverse the increase in BBB permeability, possibly reducing or eliminating the occurrence of SRS (antiepileptic therapy).

### Example of a Prophylactic Therapy

As an example of these therapeutic approaches, we consider the effects of two angiogenesis/VEGF inhibitors on the development or expression of kainic acid-induced TLE in the rat, a widely used animal model for epilepsy research. The average duration of the latent period in this model is approximately 2 weeks, and the frequency of SRSs typically stabilizes by approximately 4 weeks post-SE.

Kainic acid (KA) will be administered at a dose of 10 mg/kg via intravenous injection to male Wistar rats weighing between 280 and 320 g. In response to this injection, these animals reliably experience 4-6 hours of limbic SE, from which they emerge spontaneously. Beginning the day after SE induction, these animals will be monitored for 8 hours per day, five days per week, for the occurrence of spontaneous seizures over the course of 16 weeks. The date, time, and severity (rated according to the Racine scale) of each seizure will be recorded. Beginning one week after SE, treatment group animals will receive daily subcutaneous injections of either Cilengitide or SU5416 for 4 consecutive weeks. Cilengitide (Merck Pharmaceuticals) is an integrin inhibitor, and would therefore be considered a generic inhibitor of angiogenesis resulting from the influence of any proangiogenic growth factor. SU5416 (formally Sugen, now Pfizer Pharmaceuticals) is a specific VEGF receptor antagonist, which specifically blocks the influence of VEGF at its initial step. Both SU5416 and Cilengitide will be suspended in diluent containing 0.5% carboxymethylcellulose sodium, 0.9% sodium chloride, 0.4% polysorbate 80, and 0.9% benzyl alcohol in deionized water (Sigma-Aldrich), and will be administered at doses of 20 mg/kg and 10 mg/kg respectively. Control animals will receive equivalent volumes of diluent on the same schedule. SU5416 specifically inhibits VEGF binding to the flk-1 VEGF receptor, whereas Cilengitide inhibits integrins αvβ3 and αvβ5, which are initiated in response to VEGF (and related growth factors) and are necessary for organizing the extracellular matrix to permit angiogenesis. The effect of these treatment regimens on spontaneous seizure expression among the various experimental groups will be subsequently assessed using a repeated measures ANOVA statistical analysis.

### Example of an Antiepileptic Therapy

Kainic acid (KA) will be administered at a dose of 10 mg/kg via intravenous injection to male Wistar rats weighing between 280 and 320 g. In response to this injection, these animals reliably experience 4-6 hours of limbic SE, from which they emerge spontaneously. Beginning the day after SE induction, these animals will be monitored for 8 hours per day, five days per week, for the occurrence of spontaneous seizures over the course of 24 weeks. The date, time, and severity (rated according to the Racine scale) of each seizure will be recorded. Beginning 8 weeks after SE, treatment group animals will receive daily subcutaneous injections of either Cilengitide or SU5416, for a total of 8 weeks. Both SU5416 and Cilengitide will be suspended in diluent containing 0.5% carboxymethylcellulose sodium, 0.9% sodium chloride, 0.4% polysorbate 80, and 0.9% benzyl alcohol in deionized water (Sigma-Aldrich), and will be administered at doses of 20 mg/kg and 10 mg/kg respectively (32, 56). Control animals will receive equivalent volumes of diluent on the same schedule. SU5416 specifically inhibits VEGF binding to the flk-1 VEGF receptor, whereas Cilengitide inhibits integrins αvβ3 and αvβ5, which are initiated in response to VEGF (and related growth factors) and are necessary for organizing the extracellular matrix to permit angiogenesis. The effect of these treatment regimens on spontaneous seizure expression among the various experimental groups will be subsequently analyzed using a repeated measures ANOVA statistical design.

Measurements of BBB Permeability in KASE Animals Using Fluorescent Tracer Assays.

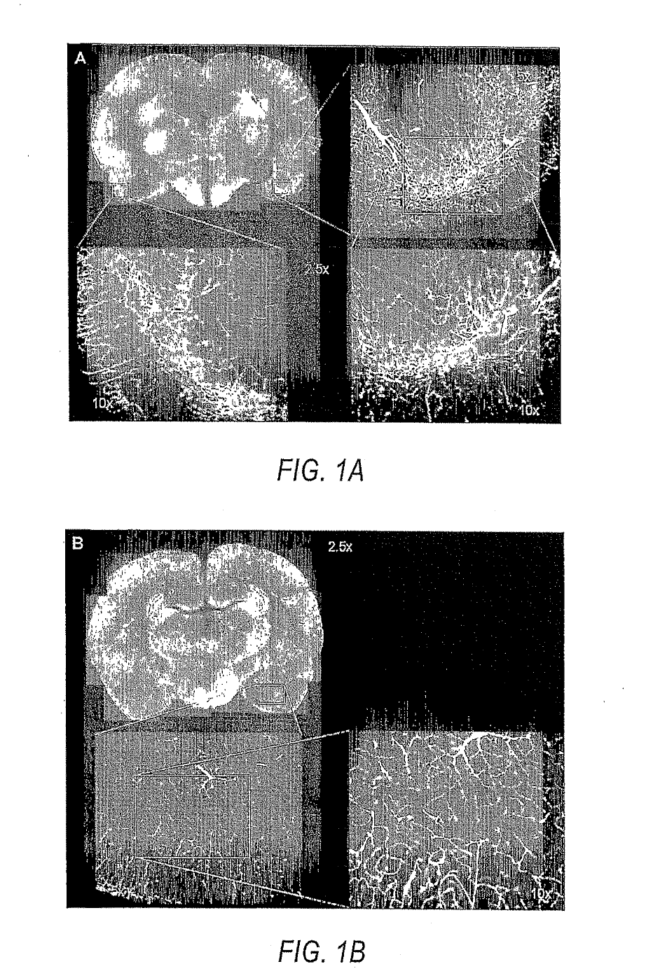

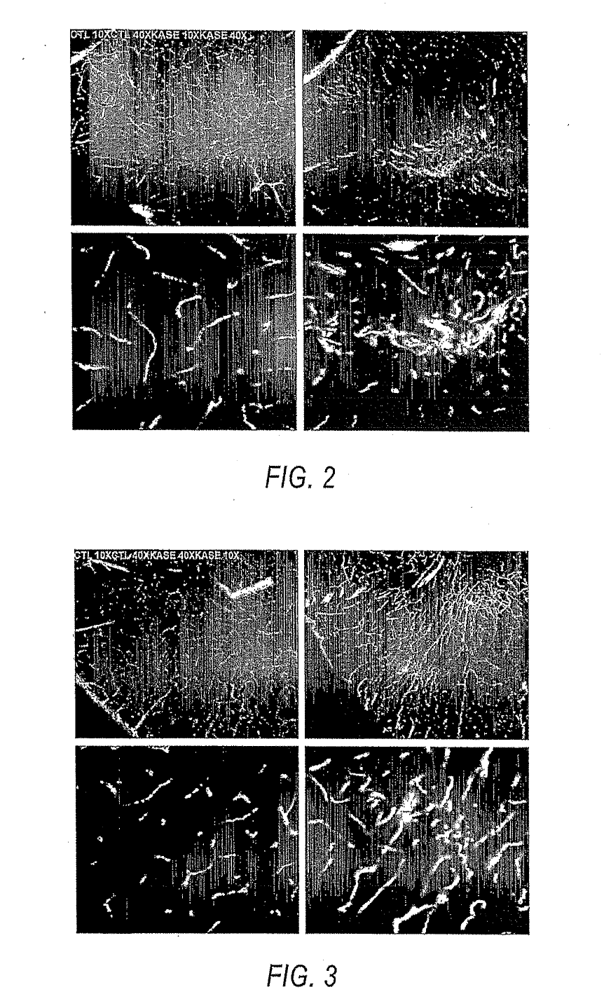

We have performed experiments using fluorescent tracers Evan's blue (designated Eb-Ald because it readily binds to the plasma protein albumin) and FITC-dextran in our kainic acid-induced status epilepticus (KASE) rats at eight, twelve, and sixteen weeks after SE induction. 100 μm sections were imaged using fluorescence microscopy (FIG. 1), and with a Nikon LSCM system. LSCM images were acquired within coronal sections sampled along the rostral-caudal axis using 10X and 40X objectives, with a scan thickness of 5 μm. Evidence of changes in microvascular plasma volume and increased blood-brain barrier (BBB) permeability were examined in multiple subfields within the amygdala, hippocampus, piriform, entorhinal and cingulate cortex, and the septum. FIG. 1 is composite EB-Alb and FITC-dextran images acquired using a fluorescent microscope from 100 μm coronal rat brain sections obtained from A) spontaneously epileptic animals 12 weeks after kainic acid-induced SE, or B) from nonepileptic age matched control animals. Regions of interest are highlighted at 5X and 10X. Note the abnormal vascular morphology and increased vascular density evident in the amygdala and piriform cortex in the post-SE epileptic brain, and the extravascular cellular uptake of EB-Alb (red) indicative of increased microvascular permeability (10X). We are presently performing the quantitative analysis of these images; however, qualitatively it is clear that microvascular plasma volume and BBB permeability are increased in several of these regions, and are most pronounced in the amygdala, hippocampus, and piriform cortex. This increase in plasma volume is associated with tortuous vascular formations where Eb-Alb is apparently leaking (FIGS. 2 and 3), as evidenced by the uptake of Eb-Alb (red) by cells in the extravascular space.

FIG. 2 is confocal EB-Alb and FITC-dextran images acquired from coronal sections at 10X (top row) and 40X (bottom row), from the ventral hippocampus of the rat brain 8 weeks after KA-induced SE (right column), together with corresponding images from an age matched control (left column). Note the vascular tangles distributed throughout the ventral CA1 and amygdala hippocampus (10X), and the extravascular cellular uptake of EB-Alb (red) indicative of increased microvascular permeability (40X) in this region. FIG. 3 is confocal EB-Alb and FITC-dextran images acquired from horizontal sections at 10X (top row) and 40X (bottom row), from the ventral hippocampus of the rat brain 16 weeks after KA-induced SE (right column), together with corresponding images from an age matched control (left column). Note the vascular tangles distributed throughout the CAl, subiculum, and entorhinal cortex (10X), and the extravascular cellular uptake of EB-Alb (red) indicative of increased microvascular permeability (40X) in this region.

All of these observations are consistent with angiogenesis and associated increases in BBB permeability resulting from SE-induced trauma.

Measurements of BBB Permeability in KASE Animals Using Radio-Iodinated Serum Albumin (RISA).

The use of RISA as a tracer for quantitative autoradiography (QAR) has been used previously to quantify regional plasma volume and BBB permeability, and yields data which is analogous to that obtained using fluorescent tracer assays. The permeability of the BBB to albumin and tracers that bind to albumin may have additional significance in the context of epilepsy, in light of a recent report implicating the leakage of serum proteins with the development of focal epileptiform activity in neocortex (16). More precise QAR measures of BBB permeability and microvascular plasma volume are obtained if a second signal can be acquired from the same tissue using a tracer confined only to the vascular compartment. This can be achieved by intravenously injecting a bolus of RISA123 near the end of the standard RISA125 perfusion interval. The short perfusion time of RISA123 prior to sacrifice prevents appreciable amounts of this material from escaping the vascular compartment, and because RISA123 has a half-life of only eight hours its emissions become undetectable within approximately 24 hours. Thus, emissions originating from the vascular compartment of a given section can be calculated by subtracting an autoradiograph obtained 5-7 days after sacrifice (produced by emissions from RISA125 alone) from one obtained immediately after sacrifice (produced by emissions from both RISA125 and RISA123).

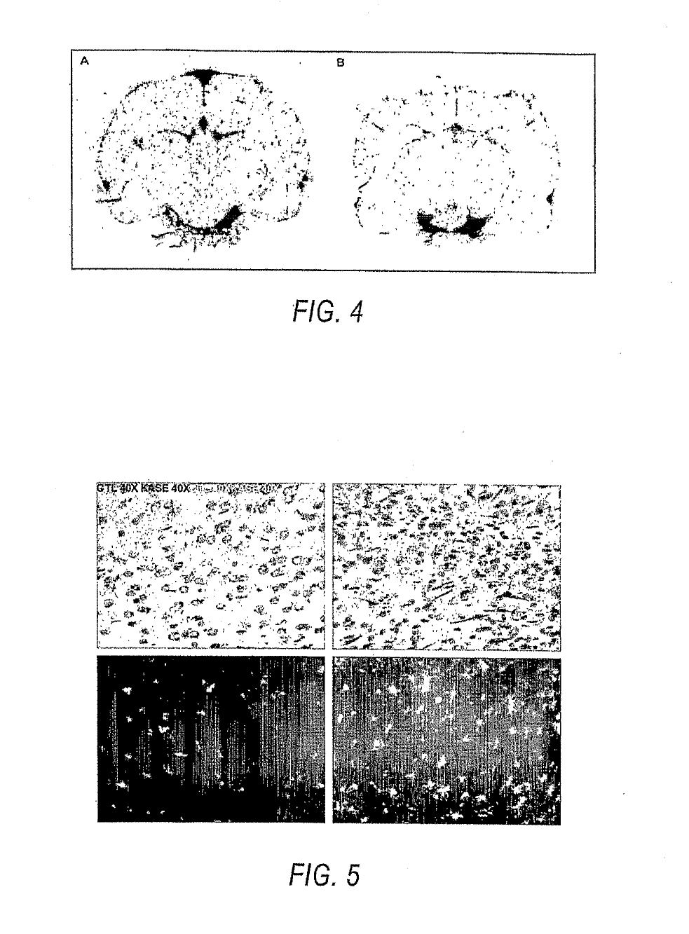

We have obtained autoradiographs reflecting the blood-to-brain distribution of RISA in the normal and spontaneously epileptic rat brain. FIG. 4 is autoradiographs obtained from coronal rat brain sections harvested twelve weeks after A) kainic acid-induced SE, or B) saline injection in an age matched control. Brains were perfused for 3 hours with RISA125 immediately prior to sacrifice. Wistar rats were given a bolus intravenous injection of RISA125, which was allowed to circulate for three hours. At the end of tracer circulation, rats were decapitated and the heads were immediately frozen in 2-methyl butane cooled to −45° C. with dry ice. Such freezing has been shown to preserve the brain morphology, blood and cerebrospinal fluid compartments, and minimize post-mortem movement of any extravascular tracers. The large dark patches on the autoradiograph reflect blood retained in large blood vessels on the pial surface of the brain, including the venous sinuses. The dark spots within the parenchyma come from blood retained in the arteries, veins, and larger microvessels with the tissue. The grainy area is produced by radiation from the smaller microvessels, and this radioactivity is used to calculate the microvascular (mostly capillaries and small venuoles) plasma volume, which we use as one index of angiogenesis, among other possible indices. Note that this granularity is increased in the spontaneously epileptic brain relative to the nonepileptic control, particularly in the ventral aspect of the brain, and is indicative of increased vascular density and/or permeability.

Immunohistochemistry for VEGF.

We have established protocols for VEGF immunohistochemistry in both frozen sectioned and formalin-fixed paraffin-embedded rat brain tissue. In our model, VEGF expression will be chronically increased following KASE and primarily localized to reactive astrocytes in regions where angiogenesis and/or increased BBB permeability are also evident. This is generally the case in brain tissue following other forms of insult, and our data obtained from regions of the brain damaged by SE are consistent with this model (FIG. 5). FIG. 5 is fluorescent (bottom row) and light microscopic (top row) images of VEGF staining acquired at 40X in the ventral hippocampus from coronal rat brain sections. Kainic acid-induced SE rats (right column), or age matched controls (left column) were sacrificed 16 weeks after KA-induced SE and processed for VEGF immunohistochemistry according to protocol. Note the presence of numerous VEGF positive reactive astrocytes in the KA-induced SE rats relative to controls and the exclusive nature of the cytosolic VEGF staining to these cells. We have observed reactive astrocytes with dramatically increased VEGF expression at 1, 2, 3, 4, 5, 6, 7, 8, and 16 weeks after KA-induced SE, localized primarily in regions which also have increased microvascular plasma volume and/or increased BBB permeability, including hippocampus, amygdala, and piriform cortex. Cytosolic expression of VEGF appears to be exclusive to reactive astrocytes in these regions, with punctate extracellular staining on adjacent neurons, astrocytes, and endothelial cells, which we interpret to be VEGF bound to flk-1 receptors.

Immunohistochemistry for Bromodeoxyuridine (BrdU).

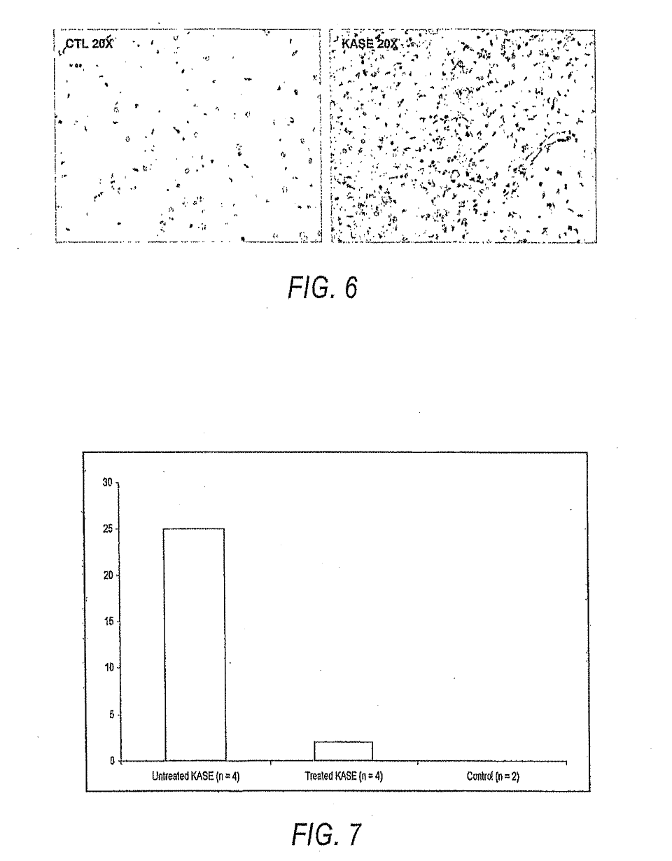

We have established protocols for BrdU immunohistochemistry in both frozen sectioned, formalin-fixed vibratome sectioned, and formalin-fixed paraffin-embedded rat brain tissue. In our model, VEGF produced by reactive astrocytes within limbic structures damaged by SE may induce localized angiogenesis. To confirm this, we injected BrdU (50 mg/kg, i.p.) for seven consecutive days in different cohorts of rats beginning 1 day, 1 week, 2 weeks, 3 weeks, and 4 weeks after SE. Animals in each cohort were sacrificed 2 weeks after completing their series of BrdU injections and processed for BrdU immunohistochemistry. During these two weeks, cells that incorporated BrdU by proliferating during the injection series are able to differentiate and migrate toward their final destinations. Since angiogenesis is generally believed to derive from proliferating endothelial cells adjacent to the site of angiogenesis, the migratory paths of these cells is presumed to be relatively short. Thus, if angiogenesis is occurring in regions damaged by SE, one would expect to find vascular elements within these regions which incorporate BrdU positive endothelial cells. We observed BrdU positive vascular elements commonly localized within limbic structures damaged by SE. Moreover, the vessels are often abnormally large and irregularly shaped, characteristic of pathological angiogenesis in the presence of high VEGF concentrations. FIG. 6 is light microscopic images of BrdU staining acquired at 20X in the amygdala from coronal rat brain sections. Kainic acid-induced SE rats (right column), or age matched controls (left column) received daily injections of BrdU (50 mg/kg, i.p.) and were sacrificed 2 weeks after completing this injection series. Note the prevalent BrdU staining within this region associated with SE. Note also the vascular affiliations of many of the BrdU positive cells and the abnormal vascular morphology indicative of pathological angiogenesis.

Effect of Angiogenesis Inhibitors on Seizure Formation In Vivo.

We have administered HET0016, an inhibitor of angiogenesis, during the “latent period” that follows KASE and precedes the emergence of spontaneous seizures, to assess whether the development of spontaneous seizures could be impeded. Optimal doses for achieving antiangiogenesis have been established for some of these compounds in other pathological contexts and we have acquired some knowledge of the progression of angiogenesis following KASE using our temporal BrdU assay. On the basis of these observations, we administered a proven antiangiogenic compound at the optimal dosage established in other pathological contexts. This compound was administered to KASE rats (n=4) via twice daily injections for fourteen consecutive days, beginning one week following KASE. Four untreated KASE rats (n=4) received sham injections of diluent and were processed in parallel. Control rats (n=2) received neither KASE nor antiangiogenic therapy. The occurrence of spontaneous seizures in these cohorts was monitored during daily during 8-hour observation sessions beginning one week following KASE, and continues to the present. The cumulative seizure data for these groups for the first three weeks of monitoring is presented in FIG. 7. The data show that treatment of KASE rats with an angiogenesis inhibitor impeded the development of spontaneous seizures relative to untreated KASE or control rats.

Each of the references identified herein is hereby incorporated by reference as though fully set forth herein.

While the present invention has been particularly shown and described with reference to the foregoing preferred and alternative embodiments, it should be understood by those skilled in the art that various alternatives to the embodiments of the invention described herein may be employed in practicing the invention without departing from the spirit and scope of the invention as defined in the following claims. It is intended that the following claims define the scope of the invention and that the method and apparatus within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

### REFERENCES

1. Hauser W A. Postscript: How should outcome be determined. in: Engel J Jr, eds. Surgical Treatment of the Epilepsies. Raven Press: New York, pp 573-579 (1987).
2. Berg A T, Shinnar S. Do seizures beget seizures? An assessment of the clinical evidence in humans. J Clin Neutophysiol 14:102-110 (1997).
3. Broadwell R D, Chariton H M, Ebert P, Hickey W F, Villegas J C, Wolf A L. Angiogenesis and the blood-brain barrier in solid and dissociated cell grafts within the CNS. Progress in Brain Research 82:95-10 (1990).
4. Maytal J, Shinnar S, Moshe S L, Alvarez L A. Low morbidity and mortality of status epilepticus in children. Pediatrics 83:323-331 (1989).
5. French J A, Williamson P D, Thadani V M, Darcy T M, Mattson R H, Spencer S S, et al. Characteristics of medial temporal lobe epilepsy: I. Results of history and physical examination. Ann Neurol 34:771-780 (1993).
6. Maher J, McLachlan R S. Febrile convulsions: is seizure duration the most important predictor of temporal lobe epilepsy? Brain 118:1521-1528 (1995).
7. Yang G Y, Betz A L. Reperfusion-induced injury to the blood-brain barrier after middle cerebral artery occlusion without craniotomy in rats. Stroke 25:1658-1665 (1994).
8. Sun D A, Sombati S, Blair R E, DeLorenzo R J. Long-lasting alterations in neuronal calcium homeostasis in an in vitro model of stroke-induced epilepsy. Cell Calcium 35:155-163 (2004).
9. Andrew R D, Fagen M, Ballyk B A, Rosen A S. Seizure susceptibility and the osmotic state. Brain Res 498:175-180 (1989).
10. Beaumont A, Marmarou A, Hayasaki K, Barzo P, Fatouros P, Corwin F, Marmarou C, Dunbar J. The permissive nature of blood brain barrier (BBB) opening in edema formation following traumatic brain injury. Acta Neurochirurgica—Supplement. 76:125-9 (2000).
11. Berg A T, Shinnar S. Unprovoked seizures in children with febrile seizures: short term outcome. Neurology 47:562-568 (1996).
12. Zhang Z G, Zhang L, Tsang W, Soltanian-Zadeh H, Morris D, Zhang R, Goussev A, Powers C, Yeich T, Chopp M. Correlation of VEGF and angiopoietin expression with disruption of blood-brain barrier and angiogenesis after focal cerebral ischemia. Journal of Cerebral Blood Flow & Metabolism. 22(4):379-92 (2002).
13. Ozawa C R, Banfi A, Glazer N L, Thurston G, Springer M L, Kraft P E, McDonald D M, Blau H M. Microenvironmental VEGF concentration, not total dose, determines a threshold between normal and aberrant angiogenesis. J Clin Invest 113:516-527 (2004).
14. Tandle A, Blazer D G, Libutti S. Antiangiogenic gene therapy of cancer: recent developments. Journal of Translational Medicine 2:22 (2004)
15. Walker M C, White H S, Sander A S. Disease modification in partial epilepsy. Brain 125:1937-1950 (2002).
16. Seiffert E, Drier J P, Ivens S, Bechmann I, Tomkins O, Hienemann U, Friedman A. Lasting blood-brain barrier disruption induces epileptic focus in the rat somatosensory cortex. J Neurosci 24:7828-7836 (2004).

1. A method of inhibiting an increase in neural microvascular permeability of a mammal, comprising the steps of:
providing an angiogenesis inhibitor that is an antagonist of at least one growth factor whose biological effects include angiogenesis in neural tissue and/or increases in neural microvascular permeability in a mammal; and
administering a pharmaceutically effective amount of the angiogenesis inhibitor to the mammal.

2-12. (canceled)
